(19) United States
(12) Patent Application Publication
(10) Pub. No.: US 2025/0145624 A1
(43) Pub. Date: May 8, 2025

(54) MEMBRANE-ASSOCIATED TYROSINE- AND THREONINE-SPECIFIC CDC2-INHIBITORY KINASE (PKMYT1) INHIBITORS AND USES THEREOF

(71) Applicant: Insilico Medicine IP Limited, Hong Kong (CN)

(72) Inventors: Xiao DING, Shanghai (CN); Fanye MENG, Shanghai (CN); Feng REN, Shanghai (CN); Yazhou WANG, Shanghai (CN); Min ZHENG, Shanghai (CN)

(21) Appl. No.: 18/837,843

(22) PCT Filed: Feb. 17, 2023
(86) PCT No.: PCT/CN2023/076827

§ 371 Date: Aug. 12, 2024

(30) Foreign Application Priority Data
Feb. 18, 2022 (WO) PCT/CN2022/076943

(51) Int. Cl. C07D 471/16; A61K 31/55; C07D 487/16

(52) U.S. Cl. CPC C07D 471/16; A61K 31/55; C07D 487/16

(57) ABSTRACT

Described herein are PKMYT1 (Myt1) inhibitors and pharmaceutical compositions comprising said inhibitors. The subject compounds and compositions are useful for the treatment of a disease or disorder associated with PKMYT1.

### CROSS-REFERENCE

This patent application claims the benefit of International Application No. PCT/CN2022/076943, filed Feb. 18, 2022; which is incorporated herein by reference in its entirety.

### BACKGROUND OF THE INVENTION

PKMYT1 (or Myt1) is a member of the Wee family and was first reported as a kinase capable of phosphorylating Cdc2 efficiently on both threonine-14 and tyrosine-15 in a Xenopus frog. PKMYT1 inhibits cell cycle progression by inhibiting the activities of cell cycle-associated proteins, such as Cyclin A, CDK1, and CDK2. PKMYT1 also drives the progression of a variety of tumors.

The inhibitory phosphorylation of cdc2 is important for the timing of entry into mitosis. Entry into mitosis is initiated by the M phase-promoting factor (MPF), a complex containing the cdc2 protein kinase and cyclin B. Proper regulation of MPF ensures that mitosis occurs only after earlier phases of the cell cycle are complete. Phosphorylation of cdc2 at Tyr-15 and Thr-14 suppresses this activity during interphase (G1, S, and G2). At G2-M transition, cdc2 is dephosphorylated at Tyr-15 and Thr-14 allowing MPF to phosphorylate its mitotic substrates.

Studies have shown that premature activation of cdc2 leads to mitotic catastrophe and cell death. Inhibition of Myt1 is predicted to cause premature activation of cdc2, and thus would kill rapidly proliferating cells. In addition, Myt1 inhibition is predicted to reduce resistance to conventional DNA-damaging chemotherapeutics, because the mechanisms by which cells avoid death involve arrest in the G2 phase of the cell cycle, and repair or DNA damage prior to division. That arrest should be prevented by blocking Myt1 inhibitory phosphorylation of cdc2. Thus forcing the cell to enter mitosis prematurely. Myt1 kinase is an important cell cycle regulator, particularly at the G2/M phase. This is due to cell cycle regulation and subsequent repair of damage to DNA or mitotic apparatus, the targets for most effective chemotherapeutic agents. Myt1 kinase offers a point of intervention downstream from these mechanisms by which tumor cells develop resistance. Inhibition of Myt1 could in and of itself have therapeutic benefit in reducing tumor proliferation, and in addition, could be used in conjunction with conventional chemotherapies to overcome drug resistance.

Based on the foregoing, there is a need to identify a potent PKMYT1 (Myt1) kinase inhibitor for the treatment of cancer.

### SUMMARY

Disclosed herein is a compound of Formula (I), or a pharmaceutically acceptable salt thereof:

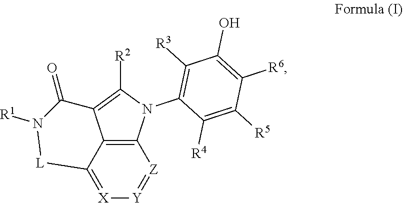

wherein:
R¹ is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
L is C₁-C₄alkylene or C₁-C₄heteroalkylene comprising 1 or 2 heteroatoms selected from the group consisting of 0, N, and S; each optionally substituted with one or more R⁷;
each R⁷ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁷ on the same atom are taken together to form an oxo;
or two R⁷ on adjacent carbons are taken together to form an alkenylene;
or two R⁷ on the same carbon are taken together to form a cycloalkyl or heterocycloalkyl, each optionally substituted with one or more R;
or two R⁷ on different atoms are taken together to form a cycloalkyl, heterocycloalkyl, aryl, or heteroaryl, each optionally substituted with one or more R;
R² is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Xa};
each R^{Xa} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Xa} on the same atom are taken together to form an oxo;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Ya};
each R^{Ya} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Ya} on the same atom are taken together to form an oxo;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
R³ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁴ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

In some embodiments of a compound of Formula (I), the compound is of Formula (Ia):

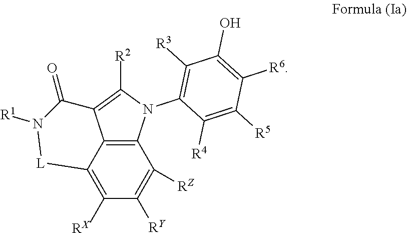

In some embodiments of a compound of Formula (I), the compound is of Formula (Ib):

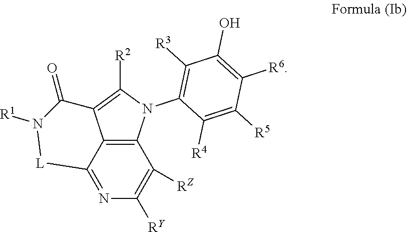

In some embodiments of a compound of Formula (I), the compound is of Formula (Ic):

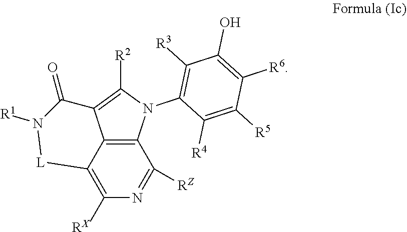

In some embodiments of a compound of Formula (I), the compound is of Formula (Id):

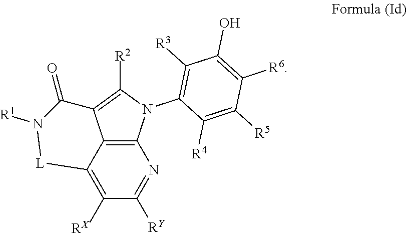

In some embodiments of a compound of Formula (I), the compound is of Formula (Ie):

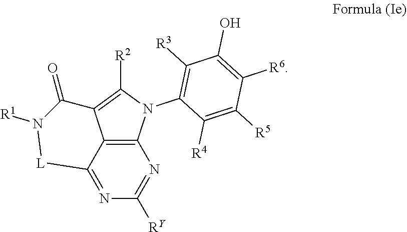

Also disclosed herein is a pharmaceutical composition comprising a compound disclosed herein, or a pharmaceutically acceptable salt thereof, and a pharmaceutically acceptable excipient.

Also disclosed herein is a method of treating cancer in a subject in need thereof, the method comprising administering to the subject a compound disclosed herein, or a pharmaceutically acceptable salt thereof.

Also disclosed herein is a method of modulating PKMYT1 in a subject, the method comprising administering to the subject the compound disclosed herein, or a pharmaceutically acceptable salt thereof.

Also disclosed herein is a method of inhibiting PKMYT1 in a subject, the method comprising administering to the subject the compound disclosed herein, or a pharmaceutically acceptable salt thereof.

Also disclosed herein is a method of selectively inhibiting PKMYT1 (e.g., over WEE1) in a subject, the method comprising administering to the subject the compound disclosed herein, or a pharmaceutically acceptable salt thereof. In some embodiments, compounds described herein selectively inhibit PKMYT1 without inhibiting WEE1. In some embodiments, the compound or a pharmaceutically acceptable salt thereof does not inhibit WEE1.

In some embodiments, the subject has cancer. In some embodiments, the cancer depends on the activity of PKMYT1. In some embodiments, the cancer overexpresses CCNE1. In some embodiments, the cancer has an inactivating mutation in the FBXW7 gene. In some embodiments, the cancer is a solid tumor. In some embodiments, the cancer is breast cancer, colorectal cancer, endometrial cancer, esophageal cancer, glioblastoma, hepatocellular carcinoma, lung cancer, neuroblastoma, ovarian cancer, prostate cancer, stomach cancer, or uterine cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:
“oxo” refers to ═O.
“Carboxyl” refers to —COOH.
“Cyano” refers to —CN.
“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀ cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₅ cycloalkyl or C₃-C₅ cycloalkenyl), or three to four carbon atoms (C₃-C₄ cycloalkyl or C₃-C₄ cycloalkenyl). In some embodiments, the cycloalkyl is a 3- to 10-membered cycloalkyl or a 3- to 10-membered cycloalkenyl. In some embodiments, the cycloalkyl is a 3- to 6-membered cycloalkyl or a 3- to 6-membered cycloalkenyl. In some embodiments, the cycloalkyl is a 5-to 6-membered cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₅ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₇ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

The terms “treat,” “treating” or “treatment,” as used herein, include alleviating, abating, or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

As used herein, a “disease or disorder associated with PKMYT1” or, alternatively, “a PKMYT1-mediated disease or disorder” means any disease or other deleterious condition in which PKMYT1, or a mutant thereof, is known or suspected to play a role.

As used herein, a “disease or disorder associated with Myt1” or, alternatively, “a Myt1-mediated disease or disorder” means any disease or other deleterious condition in which Myt1, or a mutant thereof, is known or suspected to play a role.

### Compounds

Described herein are compounds, or a pharmaceutically acceptable salt thereof useful in the treatment of a disease or disorder associated with PKMYT1.

Disclosed herein is a compound of Formula (I), or a pharmaceutically acceptable salt thereof:

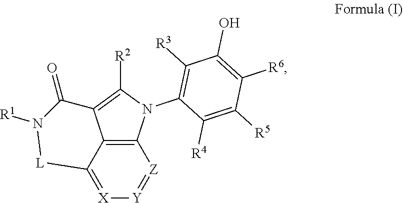

wherein:
R¹ is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
L is C₁-C₄alkylene or C₁-C₄heteroalkylene comprising 1 or 2 heteroatoms selected from the group consisting of O, N, and S; each optionally substituted with one or more R⁷;
each R⁷ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁷ on the same atom are taken together to form an oxo;
or two R⁷ on adjacent carbons are taken together to form an alkenylene;
or two R⁷ on the same carbon are taken together to form a cycloalkyl or heterocycloalkyl, each optionally substituted with one or more R;
or two R⁷ on different atoms are taken together to form a cycloalkyl, heterocycloalkyl, aryl, or heteroaryl, each optionally substituted with one or more R;
R² is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Xa};
each R^{Xa} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Xa} on the same atom are taken together to form an oxo;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Ya};
each R^{Ya} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Ya} on the same atom are taken together to form an oxo;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
R³ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁴ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

The compound of claim 1, or a pharmaceutically acceptable salt thereof, wherein the compound is of Formula (I-1):

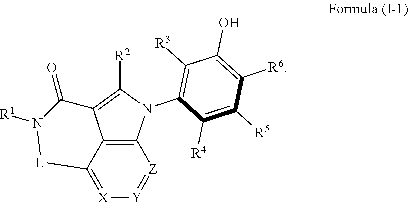

In some embodiments of a compound of Formula (I) or (I-1), X is N. In some embodiments of a compound of Formula (I) or (I-1), X is CR^{X}.

In some embodiments of a compound of Formula (I) or (I-1), Y is N. In some embodiments of a compound of Formula (I) or (I-1), Y is CR^{Y}.

In some embodiments of a compound of Formula (I) or (I-1), Z is N. In some embodiments of a compound of Formula (I) or (I-1), Z is CR^{Z}.

In some embodiments of a compound of Formula (I), the compound is of Formula (Ia):

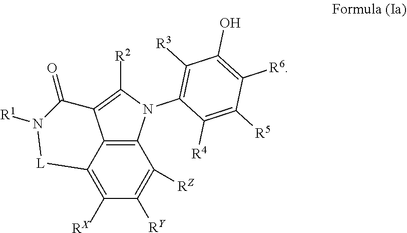

In some embodiments of a compound of Formula (I-1), the compound is of Formula (Ia-1):

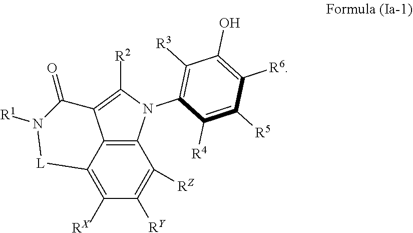

In some embodiments of a compound of Formula (I), the compound is of Formula (Ib):

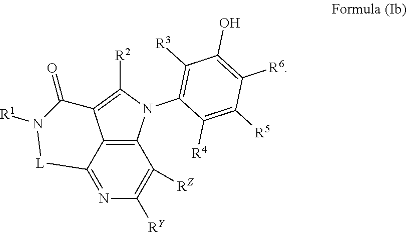

In some embodiments of a compound of Formula (I-1), the compound is of Formula (Ib-1):

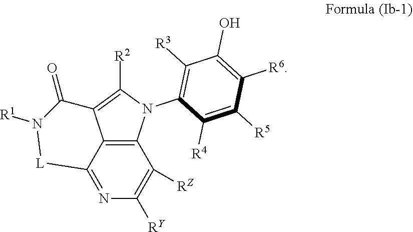

In some embodiments of a compound of Formula (I), the compound is of Formula (Ic):

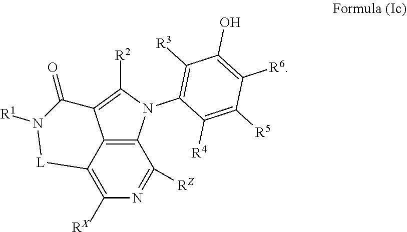

In some embodiments of a compound of Formula (I-1), the compound is of Formula (Ic-1):

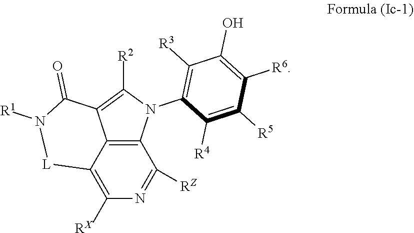

In some embodiments of a compound of Formula (I), the compound is of Formula (Id):

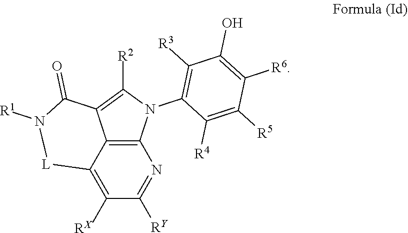

In some embodiments of a compound of Formula (I-1), the compound is of Formula (Id-1):

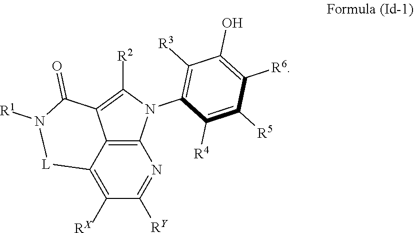

In some embodiments of a compound of Formula (I), the compound is of Formula (Ie):

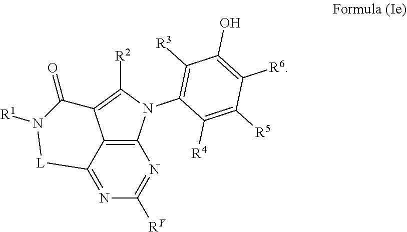

In some embodiments of a compound of Formula (I-1), the compound is of Formula (Ie-1):

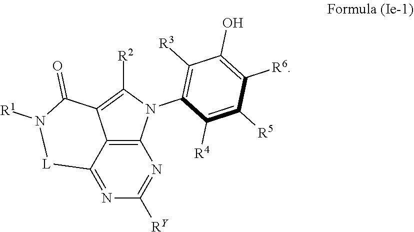

In some embodiments of a compound of Formula (I), (I-1), (Ia)-(Ie), or (Ia-1)-(Ie-1), R³ is halogen, C₁-C₆alkyl, or C₁-C₆haloalkyl. In some embodiments of a compound of Formula (I), (I-1), (Ia)-(Ie), or (Ia-1)-(Ie-1), R³ is halogen or C₁-C₆alkyl. In some embodiments of a compound of Formula (I), (I-1), (Ia)-(Ie), or (Ia-1)-(Ie-1), R³ is C₁-C₆alkyl. In some embodiments of a compound of Formula (I), (I-1), (Ia)-(Ie), or (Ia-1)-(Ie-1), R³ is C₁-C₃alkyl. In some embodiments of a compound of Formula (I), (I-1), (Ia)-(Ie), or (Ia-1)-(Ie-1), R³ is n-propyl or isopropyl. In some embodiments of a compound of Formula (I), (I-1), (Ia)-(Ie), or (Ia-1)-(Ie-1), R³ is ethyl. In some embodiments of a compound of Formula (I), (I-1), (Ia)-(Ie), or (Ia-1)-(Ie-1), R³ is methyl.

In some embodiments of a compound of Formula (I), (I-1), (Ia)-(Ie), or (Ia-1)-(Ie-1), R⁴ is halogen, C₁-C₆alkyl, or C₁-C₆haloalkyl. In some embodiments of a compound of Formula (I), (I-1), (Ia)-(Ie), or (Ia-1)-(Ie-1), R⁴ is halogen or C₁-C₆alkyl. In some embodiments of a compound of Formula (I), (I-1), (Ia)-(Ie), or (Ia-1)-(Ie-1), R⁴ is C₁-C₆alkyl. In some embodiments of a compound of Formula (I), (I-1), (Ia)-(Ie), or (Ia-1)-(Ie-1), R⁴ is C₁-C₃alkyl. In some embodiments of a compound of Formula (I), (I-1), (Ia)-(Ie), or (Ia-1)-(Ie-1), R⁴ is n-propyl or isopropyl. In some embodiments of a compound of Formula (I), (I-1), (Ia)-(Ie), or (Ia-1)-(Ie-1), R⁴ is ethyl. In some embodiments of a compound of Formula (I), (I-1), (Ia)-(Ie), or (Ia-1)-(Ie-1), R⁴ is methyl.

In some embodiments of a compound of Formula (I), (I-1), (Ia)-(Ie), or (Ia-1)-(Ie-1), R⁵ is hydrogen, halogen, C₁-C₆alkyl, or C₁-C₆haloalkyl. In some embodiments of a compound of Formula (I), (I-1), (Ia)-(Ie), or (Ia-1)-(Ie-1), R⁵ is hydrogen, halogen, C₁-C₃alkyl, or C₁-C₃haloalkyl. In some embodiments of a compound of Formula (I), (I-1), (Ia)-(Ie), or (Ia-1)-(Ie-1), R⁵ is hydrogen.

In some embodiments of a compound of Formula (I), (I-1), (Ia)-(Ie), or (Ia-1)-(Ie-1), R⁶ is hydrogen, halogen, C₁-C₆alkyl, or C₁-C₆haloalkyl. In some embodiments of a compound of Formula (I), (I-1), (Ia)-(Ie), or (Ia-1)-(Ie-1), R⁶ is hydrogen, halogen, C₁-C₃alkyl, or C₁-C₃haloalkyl. In some embodiments of a compound of Formula (I), (I-1), (Ia)-(Ie), or (Ia-1)-(Ie-1), R⁶ is hydrogen.

In some embodiments of a compound of Formula (I), (I-1), (Ia)-(Ie), or (Ia-1)-(Ie-1), R² is halogen, —CN, —OH, —OR^{a}, —NR^{c}R^{d}, C₁-C₆alkyl, or C₁-C₆haloalkyl. In some embodiments of a compound of Formula (I), (I-1), (1a)-(Ie), or (Ia-1)-(Ie-1), R² is —OH, —OR^{a}, or —NR^{c}R^{d}. In some embodiments of a compound of Formula (I), (I-1), (Ia)-(Ie), or (Ia-1)-(Ie-1), R² is —NR^{c}R^{d}. In some embodiments of a compound of Formula (I), (I-1), (Ia)-(Ie), or (Ia-1)-(Ie-1), R² is —NH(C₁-C₆alkyl). In some embodiments of a compound of Formula (I), (I-1), (Ia)-(Ie), or (Ia-1)-(Ie-1), R² is NH₂.

In some embodiments of a compound of Formula (I), (I-1), (Ia), (Ic), (Id), (Ia-1), (Ic-1), or (Id-1), R^{X} is hydrogen, halogen, —CN, —OH, —OR^{a}, —NR^{c}R^{d}, —C(═O)R^{a}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{Xa}. In some embodiments of a compound of Formula (I), (I-1), (Ia), (Ic), (Id), (Ia-1), (Ic-1), or (Id-1), R^{X} is hydrogen, halogen, —CN, —OH, —OR^{a}, —NR^{c}R^{d}, —C(═O)R^{a}, C₁-C₆alkyl, C₁-C₆haloalkyl, cycloalkyl, heterocycloalkyl, aryl, or heteroaryl; wherein the alkyl, cycloalkyl, heterocycloalkyl, aryl, and heteroaryl is optionally substituted with one or more R^{Xa}. In some embodiments of a compound of Formula (I), (I-1), (Ia), (Ic), (Id), (Ia-1), (Ic-1), or (Id-1), R^{X} is hydrogen, halogen, —CN, —C(═O)R^{a}, C₁-C₆alkyl, heterocycloalkyl, or heteroaryl; wherein the alkyl, heterocycloalkyl, and heteroaryl is optionally substituted with one or more R^{Xa}. In some embodiments of a compound of Formula (I), (I-1), (Ia), (Ic), (Id), (Ia-1), (Ic-1), or (Id-1), R^{X} is optionally substituted aryl or optionally substituted heteroaryl. In some embodiments of a compound of Formula (I), (I-1), (Ia), (Ic), (Id), (Ia-1), (Ic-1), or (Id-1), R^{X} is optionally substituted 5 or 6-membered heteroaryl. In some embodiments of a compound of Formula (I), (I-1), (Ia), (Ic), (Id), (Ia-1), (Ic-1), or (Id-1), R^{X} is optionally substituted heteroaryl containing 1-3 nitrogens and 0-1 oxygen or sulfur. In some embodiments of a compound of Formula (I), (I-1), (Ia), (Ic), (Id), (Ia-1), (Ic-1), or (Id-1), R^{X} is optionally substituted cycloalkyl or heterocycloalkyl. In some embodiments of a compound of Formula (I), (I-1), (Ia), (Ic), (Id), (Ia-1), (Ic-1), or (Id-1), R^{X} is optionally substituted 4- to 6-membered heterocycloalkyl. In some embodiments of a compound of Formula (I), (I-1), (Ia), (Ic), (Id), (Ia-1), (Ic-1), or (Id-1), R^{X} is optionally substituted 4-membered heterocycloalkyl. In some embodiments of a compound of Formula (I), (I-1), (Ia), (Ic), (Id), (Ia-1), (Ic-1), or (Id-1), R^{X} is optionally substituted 5-membered heterocycloalkyl. In some embodiments of a compound of Formula (I), (I-1), (Ia), (Ic), (Id), (Ia-1), (Ic-1), or (Id-1), R^{X} is optionally substituted 6-membered heterocycloalkyl. In some embodiments of a compound of Formula (I), (I-1), (Ia), (Ic), (Id), (Ia-1), (Ic-1), or (Id-1), R^{X} is hydrogen, halogen, or C₁-C₆alkyl. In some embodiments of a compound of Formula (I), (I-1), (Ia), (Ic), (Id), (Ia-1), (Ic-1), or (Id-1), R^{X} is hydrogen, halogen, or C₁-C₃alkyl. In some embodiments of a compound of Formula (I), (I-1), (Ia), (Ic), (Id), (Ia-1), (Ic-1), or (Id-1), R^{X} is halogen or C₁-C₆alkyl. In some embodiments of a compound of Formula (I), (I-1), (Ia), (Ic), (Id), (Ia-1), (Ic-1), or (Id-1), R^{X} is halogen or C₁-C₃alkyl. In some embodiments of a compound of Formula (I), (I-1), (Ia), (Ic), (Id), (Ia-1), (Ic-1), or (Id-1), R^{X} is C₁-C₆alkyl. In some embodiments of a compound of Formula (I), (I-1), (Ia), (Ic), (Id), (Ia-1), (Ic-1), or (Id-1), R^{X} is C₁-C₃alkyl. In some embodiments of a compound of Formula (I), (I-1), (Ia), (Ic), (Id), (Ia-1), (Ic-1), or (Id-1), R^{X} is methyl. In some embodiments of a compound of Formula (I), (I-1), (Ia), (Ic), (Id), (Ia-1), (Ic-1), or (Id-1), R^{X} is —CN. In some embodiments of a compound of Formula (I), (I-1), (Ia), (Ic), (Id), (Ia-1), (Ic-1), or (Id-1), R^{X} is —C(═O)Me.

In some embodiments of a compound of Formula (I), (I-1), (Ia), (Ic), (Id), (Ia-1), (Ic-1), or (Id-1); each R^{Xa} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆heteroalkyl, cycloalkyl, or heterocycloalkyl; wherein each alkyl, cycloalkyl, and heterocycloalkyl is independently optionally substituted with one or more R; or two R^{Xa} on the same atom are taken together to form an oxo. In some embodiments of a compound of Formula (I), (I-1), (Ia), (Ic), (Id), (Ia-1), (Ic-1), or (Id-1); each R^{Xa} is independently halogen, —CN, —OH, —OR^{a}, —NR^{c}R^{d}, C₁-C₆alkyl, C₁-C₆haloalkyl, or C₁-C₆heteroalkyl; wherein each alkyl is independently optionally substituted with one or more R; or two R^{Xa} on the same atom are taken together to form an oxo. In some embodiments of a compound of Formula (I), (I-1), (Ia), (Ic), (Id), (Ia-1), (Ic-1), or (Id-1); each R^{Xa} is independently halogen, —OH, —OR^{a}, C₁-C₆alkyl, or C₁-C₆heteroalkyl; wherein each alkyl is independently optionally substituted with one or more R; or two R^{Xa} on the same atom are taken together to form an oxo. In some embodiments of a compound of Formula (I), (I-1), (Ia), (Ic), (Id), (Ia-1), (Ic-1), or (Id-1); each R^{Xa} is independently halogen, —OH, —OR^{a}, C₁-C₆alkyl, or C₁-C₆heteroalkyl; or two R^{Xa} on the same atom are taken together to form an oxo. In some embodiments of a compound of Formula (I), (I-1), (Ia), (Ic), (Id), (Ia-1), (Ic-1), or (Id-1); each R^{Xa} is independently —OR^{a}, C₁-C₆alkyl, or C₁-C₆heteroalkyl; or two R^{Xa} on the same atom are taken together to form an oxo.

In some embodiments of a compound of Formula (I), (I-1), (Ia), (Ic), (Id), (Ia-1), (Ic-1), or (Id-1), R^{X} is

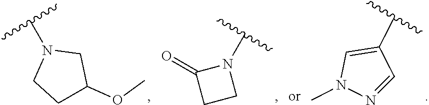

In some embodiments of a compound of Formula (I), (I-1), (Ia), (Ib), (Id), (Ie) (Ia-1), (Ib-1), (Id-1), or (Ie), R^{Y} is hydrogen, halogen, —CN, —OH, —OR^{a}, —NR^{c}R^{d}, C₁-C₆alkyl, C₁-C₆haloalkyl, cycloalkyl, or heterocycloalkyl; wherein the alkyl, cycloalkyl, and heterocycloalkyl is optionally substituted with one or more R^{Ya}. In some embodiments of a compound of Formula (I), (I-1), (Ia), (Ib), (Id), (Ie) (Ia-1), (Ib-1), (Id-1), or (Ie), R^{Y} is hydrogen, halogen, C₁-C₆alkyl, or heterocycloalkyl; wherein the alkyl and heterocycloalkyl is optionally substituted with one or more R^{Ya}. In some embodiments of a compound of Formula (I), (I-1), (Ia), (Ib), (Id), (Ie) (Ia-1), (Ib-1), (Id-1), or (Ie), R^{Y} is hydrogen, C₁-C₆alkyl or heterocycloalkyl; wherein the alkyl and heterocycloalkyl is optionally substituted with one or more R^{Ya}. In some embodiments of a compound of Formula (I), (I-1), (Ia), (Ib), (Id), (Ie) (Ia-1), (Ib-1), (Id-1), or (Ie), R^{Y} is heterocycloalkyl optionally substituted with one or more R^{Ya}. In some embodiments of a compound of Formula (I), (I-1), (Ia), (Ib), (Id), (Ie) (Ia-1), (Ib-1), (Id-1), or (Ie), R^{Y} is 5-membered ring. In some embodiments of a compound of Formula (I), (I-1), (Ia), (Ib), (Id), (Ie) (Ia-1), (Ib-1), (Id-1), or (Ie), R^{Y} is 6-membered ring. In some embodiments of a compound of Formula (I), (I-1), (Ia), (Ib), (Id), (Ie) (Ia-1), (Ib-1), (Id-1), or (Ie), R^{Y} is hydrogen, halogen, —CN, —OH, C₁-C₆alkyl, C₁-C₆haloalkyl, cycloalkyl, or heterocycloalkyl; wherein the alkyl, cycloalkyl, and heterocycloalkyl is optionally substituted with one or more R^{Ya}. In some embodiments of a compound of Formula (I), (I-1), (Ia), (Ib), (Id), (Ie) (Ia-1), (Ib-1), (Id-1), or (Ie), R^{Y} is hydrogen, halogen, C₁-C₆alkyl, or C₁-C₆haloalkyl. In some embodiments of a compound of Formula (I), (I-1), (Ia), (Ib), (Id), (Ie) (Ia-1), (Ib-1), (Id-1), or (Ie), R^{Y} is hydrogen or C₁-C₆alkyl. In some embodiments of a compound of Formula (I), (I-1), (Ia), (Ib), (Id), (Ie) (Ia-1), (Ib-1), (Id-1), or (Ie), R^{Y} is hydrogen or C₁-C₃alkyl. In some embodiments of a compound of Formula (I), (I-1), (Ia), (Ib), (Id), (Ie) (Ia-1), (Ib-1), (Id-1), or (Ie), R^{Y} is hydrogen. In some embodiments of a compound of Formula (I), (I-1), (Ia), (Ib), (Id), (Ie) (Ia-1), (Ib-1), (Id-1), or (Ie), R^{Y} is C₁-C₆alkyl. In some embodiments of a compound of Formula (I), (I-1), (Ia), (Ib), (Id), (Ie) (Ia-1), (Ib-1), (Id-1), or (Ie), R^{Y} is C₁-C₃alkyl. In some embodiments of a compound of Formula (I), (I-1), (Ia), (Ib), (Id), (Ie) (Ia-1), (Ib-1), (Id-1), or (Ie), R^{Y} is methyl. In some embodiments of a compound of Formula (I), (I-1), (Ia), (Ib), (Id), (Ie) (Ia-1), (Ib-1), (Id-1), or (Ie), R^{Y} is C₁-C₆haloalkyl. In some embodiments of a compound of Formula (I), (I-1), (Ia), (Ib), (Id), (Ie) (Ia-1), (Ib-1), (Id-1), or (Ie), R^{Y} is C₁-C₃haloalkyl. In some embodiments of a compound of Formula (I), (I-1), (Ia), (Ib), (Id), (Ie) (Ia-1), (Ib-1), (Id-1), or (Ie), R^{Y} is CF₃.

In some embodiments of a compound of Formula (I), (I-1), (Ia), (Ib), (Id), (Ie) (Ia-1), (Ib-1), (Id-1), or (Ie), each R^{Ya} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R; or two R^{Ya} on the same atom are taken together to form an oxo. In some embodiments of a compound of Formula (I), (I-1), (Ia), (Ib), (Id), (Ie) (Ia-1), (Ib-1), (Id-1), or (Ie), each R^{Ya} is independently halogen, —CN, —OH, —OR^{a}, —NR^{c}R^{d}, C₁-C₆alkyl, C₁-C₆haloalkyl, or C₁-C₆heteroalkyl; or two R^{Ya} on the same atom are taken together to form an oxo. In some embodiments of a compound of Formula (I), (I-1), (Ia), (Ib), (Id), (Ie) (Ia-1), (Ib-1), (Id-1), or (Ie), each R^{Ya} is independently halogen, —OH, —OR^{a}, —NR^{c}R^{d}, C₁-C₆alkyl, or C₁-C₆haloalkyl; or two R^{Ya} on the same atom are taken together to form an oxo. In some embodiments of a compound of Formula (I), (I-1), (Ia), (Ib), (Id), (Ie) (Ia-1), (Ib-1), (Id-1), or (Ie), one or more R^{Ya} is independently halogen, —OH, —OW, —NR^{c}R^{d}, optionally substituted C₁-C₆alkyl, optionally substituted C₁-C₆haloalkyl, or optionally substituted C₁-C₆heteroalkyl; or two R^{Ya} on the same atom are taken together to form an oxo. In some embodiments of a compound of Formula (I), (I-1), (Ia), (Ib), (Id), (Ie) (Ia-1), (Ib-1), (Id-1), or (Ie), one or more R^{Ya} is —OR^{a}. In some embodiments of a compound of Formula (I), (I-1), (Ia), (Ib), (Id), (Ie) (Ia-1), (Ib-1), (Id-1), or (Ie), one or more R^{Ya} is optionally substituted C₁-C₆heteroalkyl.

In some embodiments of a compound of Formula (I), (I-1), (Ia)-(Ic), or (Ia-1)-(Ic-1), R^{Z} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R. In some embodiments of a compound of Formula (I), (I-1), (Ia)-(Ic), or (Ia-1)-(Ic-1), R^{Z} is hydrogen, halogen, —CN, —OH, —OR^{a}, —NR^{c}R^{d}, C₁-C₆alkyl, or C₁-C₆haloalkyl; wherein the alkyl is optionally substituted with one or more R. In some embodiments of a compound of Formula (I), (I-1), (Ia)-(Ic), or (Ia-1)-(Ic-1), R^{Z} is hydrogen, halogen, —CN, —OH, —OR^{a}, —NR^{c}R^{d}, C₁-C₆alkyl, or C₁-C₆haloalkyl. In some embodiments of a compound of Formula (I), (I-1), (Ia)-(Ic), or (Ia-1)-(Ic-1), R^{Z} is hydrogen, halogen, —CN, —OH, C₁-C₆alkyl, C₁-C₆haloalkyl, cycloalkyl, or heterocycloalkyl. In some embodiments of a compound of Formula (I), (I-1), (Ia)-(Ic), or (Ia-1)-(Ic-1), R^{Z} is hydrogen, halogen, C₁-C₆alkyl, or C₁-C₆haloalkyl. In some embodiments of a compound of Formula (I), (I-1), (Ia)-(Ic), or (Ia-1)-(Ic-1), R^{Z} is hydrogen, halogen, or C₁-C₆alkyl. In some embodiments of a compound of Formula (I), (I-1), (Ia)-(Ic), or (Ia-1)-(Ic-1), R^{Z} is hydrogen, halogen, or C₁-C₃alkyl. In some embodiments of a compound of Formula (I), (I-1), (Ia)-(Ic), or (Ia-1)-(Ic-1), R^{Z} is hydrogen or C₁-C₃alkyl. In some embodiments of a compound of Formula (I), (I-1), (Ia)-(Ic), or (Ia-1)-(Ic-1), R^{Z} is hydrogen.

In some embodiments of a compound of Formula (I), (I-1), (Ia)-(Ie), or (Ia-1)-(Ie-1), R¹ is hydrogen or C₁-C₆alkyl. In some embodiments of a compound of Formula (I), (I-1), (Ia)-(Ie), or (Ia-1)-(Ie-1), R¹ is hydrogen or C₁-C₃alkyl. In some embodiments of a compound of Formula (I), (I-1), (Ia)-(Ie), or (Ia-1)-(Ie-1), R¹ is hydrogen. In some embodiments of a compound of Formula (I), (I-1), (Ia)-(Ie), or (Ia-1)-(Ie-1), R¹ is methyl.

In some embodiments of a compound of Formula (I), (I-1), (Ia)-(Ie), or (Ia-1)-(Ie-1), L is C₁-C₄alkylene optionally substituted with one or more R⁷. In some embodiments of a compound of Formula (I), (I-1), (Ia)-(Ie), or (Ia-1)-(Ie-1), L is C₁-C₃alkylene optionally substituted with one or more R⁷. In some embodiments of a compound of Formula (I), (I-1), (Ia)-(Ie), or (Ia-1)-(Ie-1), L is C₂alkylene optionally substituted with one or more R⁷. In some embodiments of a compound of Formula (I), (I-1), (Ia)-(Ie), or (Ia-1)-(Ie-1), L is C₂alkylene.

In some embodiments of a compound of Formula (I), (I-1), (Ia)-(Ie), or (Ia-1)-(Ie-1), L is C₁-C₄heteroalkylene comprising 1 or 2 heteroatoms selected from the group consisting of O, N, and S; optionally substituted with one or more R⁷. In some embodiments of a compound of Formula (I), (I-1), (Ia)-(Ie), or (Ia-1)-(Ie-1), L is C₁-C₃heteroalkylene comprising 1 or 2 heteroatoms selected from the group consisting of O, N, and S; optionally substituted with one or more R⁷. In some embodiments of a compound of Formula (I), (I-1), (Ia)-(Ie), or (Ia-1)-(Ie-1), L is C₁-C₂heteroalkylene comprising 1 heteroatom selected from the group consisting of O, N, and S; optionally substituted with one or more R⁷. In some embodiments of a compound of Formula (I), (I-1), (Ia)-(Ie), or (Ia-1)-(Ie-1), L is C₁-C₂heteroalkylene comprising 1 heteroatom selected from the group consisting of O and N; optionally substituted with one or more R⁷. In some embodiments of a compound of Formula (I), (I-1), (Ia)-(Ie), or (Ia-1)-(Ie-1), L is C₁-C₃heteroalkylene comprising 1 heteroatom that is O; optionally substituted with one or more R⁷. In some embodiments of a compound of Formula (I), (I-1), (Ia)-(Ie), or (Ia-1)-(Ie-1), L is C₃heteroalkylene comprising 1 heteroatom that is O; optionally substituted with one or more R⁷. In some embodiments of a compound of Formula (I), (I-1), (Ia)-(Ie), or (Ia-1)-(Ie-1), L is C₁-C₂heteroalkylene comprising 1 heteroatom that is O; optionally substituted with one or more R⁷.

In some embodiments of a compound of Formula (I), (I-1), (Ia)-(Ie), or (Ia-1)-(Ie-1), each R⁷ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R; or two R⁷ on the same atom are taken together to form an oxo; or two R⁷ on adjacent carbons are taken together to form an alkenylene. In some embodiments of a compound of Formula (I), (I-1), (Ia)-(Ie), or (Ia-1)-(Ie-1), each R⁷ is independently halogen, —CN, —OH, —OR^{a}, —NR^{c}R^{d}, C₁-C₆alkyl, or C₁-C₆haloalkyl; wherein each alkyl is independently optionally substituted with one or more R. In some embodiments of a compound of Formula (I), (I-1), (Ia)-(Ie), or (Ia-1)-(Ie-1), each R⁷ is independently halogen, —CN, —OH, —OR^{a}, —NR^{c}R^{d}, C₁-C₆alkyl, or C₁-C₆haloalkyl. In some embodiments of a compound of Formula (I), (I-1), (Ia)-(Ie), or (Ia-1)-(Ie-1), each R⁷ is independently halogen, —CN, —OH, —OR^{a}, or —NR^{c}R^{d}. In some embodiments of a compound of Formula (I), (I-1), (Ia)-(Ie), or (Ia-1)-(Ie-1), each R⁷ is independently halogen. In some embodiments of a compound of Formula (I), (I-1), (Ia)-(Ie), or (Ia-1)-(Ie-1), two R⁷ on the same carbon are taken together to form an oxo. In some embodiments of a compound of Formula (I), (I-1), (Ia)-(Ie), or (Ia-1)-(Ie-1), two R⁷ on adjacent carbons are taken together to form an alkenylene.

In some embodiments of a compound of Formula (I), (I-1), (Ia)-(Ie), or (Ia-1)-(Ie-1), two R⁷ on the same carbon are taken together to form a cycloalkyl or heterocycloalkyl, each optionally substituted with one or more R. In some embodiments of a compound of Formula (I), (I-1), (Ia)-(Ie), or (Ia-1)-(Ie-1), two R⁷ on the same carbon are taken together to form a cycloalkyl optionally substituted with one or more R. In some embodiments of a compound of Formula (I), (I-1), (Ia)-(Ie), or (Ia-1)-(Ie-1), two R⁷ on the same carbon are taken together to form a heterocycloalkyl, each optionally substituted with one or more R.

In some embodiments of a compound of Formula (I), (I-1), (Ia)-(Ie), or (Ia-1)-(Ie-1), two R⁷ on different atoms are taken together to form a cycloalkyl, heterocycloalkyl, aryl, or heteroaryl, each optionally substituted with one or more R. In some embodiments of a compound of Formula (I), (I-1), (Ia)-(Ie), or (Ia-1)-(Ie-1), two R⁷ on different atoms are taken together to form a cycloalkyl optionally substituted with one or more R. In some embodiments of a compound of Formula (I), (I-1), (Ia)-(Ie), or (Ia-1)-(Ie-1), two R⁷ on different atoms are taken together to form a heterocycloalkyl optionally substituted with one or more R. In some embodiments of a compound of Formula (I), (I-1), (Ia)-(Ie), or (Ia-1)-(Ie-1), two R⁷ on different atoms are taken together to form an aryl optionally substituted with one or more R. In some embodiments of a compound of Formula (I), (I-1), (Ia)-(Ie), or (Ia-1)-(Ie-1), two R⁷ on different atoms are taken together to form an heteroaryl optionally substituted with one or more R.

In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, cycloalkyl, heterocycloalkyl, aryl, and heteroaryl is independently optionally substituted with one or more R. In some embodiments of a compound disclosed herein, each R^{a} is independently C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, each R^{a} is independently C₁-C₆alkyl, C₁-C₆haloalkyl, or cycloalkyl, heterocycloalkyl. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, cycloalkyl, heterocycloalkyl, aryl, and heteroaryl is independently optionally substituted with one or more R. In some embodiments of a compound disclosed herein, each R^{b} is independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, each R^{b} is independently hydrogen, C₁-C₆alkyl, C₁-C₆haloalkyl, or cycloalkyl, heterocycloalkyl. In some embodiments of a compound disclosed herein, each R^{b} is independently hydrogen, C₁-C₆alkyl or C₁-C₆haloalkyl. In some embodiments of a compound disclosed herein, each R^{b} is independently hydrogen or C₁-C₆alkyl. In some embodiments of a compound disclosed herein, each R^{b} is hydrogen. In some embodiments of a compound disclosed herein, each R^{b} is independently C₁-C₆alkyl.

In some embodiments of a compound disclosed herein, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, cycloalkyl, heterocycloalkyl, aryl, and heteroaryl is independently optionally substituted with one or more R. In some embodiments of a compound disclosed herein, each R^{c} and R^{d} are independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, each R^{c} and R^{d} are independently hydrogen, C₁-C₆alkyl, C₁-C₆haloalkyl, or cycloalkyl, heterocycloalkyl. In some embodiments of a compound disclosed herein, each R^{c} and R^{d} are independently hydrogen, C₁-C₆alkyl or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are hydrogen. In some embodiments of a compound disclosed herein, each R^{c} and R^{d} are independently C₁-C₆alkyl.

In some embodiments of a compound disclosed herein, R^{c} and R^{d} are taken together with the atom to which they are attached to form a heterocycloalkyl optionally substituted with one or more R.

In some embodiments of a compound disclosed herein, each R is independently halogen, —CN, —OH, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl; or two R on the same atom form an oxo. In some embodiments of a compound disclosed herein, each R is independently halogen, —CN, —OH, —NH₂, —NHCH₃, —N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl; or two R on the same atom form an oxo. In some embodiments of a compound disclosed herein, each R is independently halogen, —CN, —OH, C₁-C₆alkyl, C₁-C₆alkoxy, or C₁-C₆haloalkyl; or two R on the same atom form an oxo. In some embodiments of a compound disclosed herein, each R is independently halogen, —CN, —OH, or C₁-C₆alkyl; or two R on the same atom form an oxo. In some embodiments of a compound disclosed herein, each R is independently halogen, —OH, or C₁-C₆alkyl. In some embodiments of a compound disclosed herein, each R is independently halogen or C₁-C₆alkyl. In some embodiments of a compound disclosed herein, each R is independently halogen.

In some embodiments of a compound disclosed herein, one or more of R, R¹, R², R³, R⁴, R⁵, R⁶, R⁷, R^{X}, R^{Xa}, R^{Y}, R^{Ya}, R^{Z}, R^{a}, R^{b}, R^{c}, and R^{d} groups comprise deuterium at a percentage higher than the natural abundance of deuterium.

In some embodiments of a compound disclosed herein, one or more ¹H are replaced with one or more deuteriums in one or more of the following groups R, R¹, R², R³, R⁴, R⁵, R⁶, R⁷, R^{X}, R^{Xa}, R^{Y}, R^{Ya}, R^{Z}, R^{a}, R^{b}, R^{c}, and R^{d}.

In some embodiments of a compound disclosed herein, the abundance of deuterium in each of R, R¹, R², R³, R⁴, R⁵, R⁶, R⁷, R^{X}, R^{Xa}, R^{Y}, R^{Ya}, R^{Z}, R^{a}, R^{b}, R^{c}, and R^{d} is independently at least 1%, at least 10%, at least 20%, at least 30%, at least 40%, at least 50%, at least 60%, at least 70%, at least 80%, at least 90%, or 100% by molar.

Any combination of the groups described above for the various variables is contemplated herein. Throughout the specification, groups and substituents thereof are chosen by one skilled in the field to provide stable moieties and compounds.

In some embodiments the compound disclosed herein, or a pharmaceutically acceptable salt thereof, is one of the compounds in Table 1.

TABLE 1

| Example | Structure |
|---|---|
| 1 | 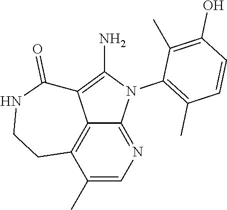 [SMILES: Cc1ccc(O)c(C)c1-n1c(N)c2c3c(c(C)cnc31)CCNC2=O] |
| 2 | 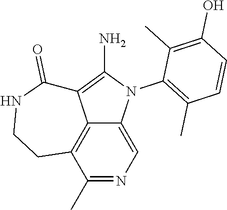 [SMILES: Cc1ccc(O)c(C)c1-n1c(N)c2c3c(c(C)ncc31)CCNC2=O] |
| 3 | 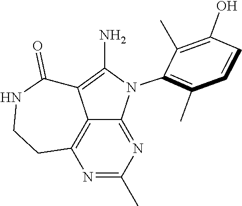 |

In some embodiments the compound disclosed herein, or a pharmaceutically acceptable salt thereof, is one of the compounds in Table 2.

TABLE 2
Structure

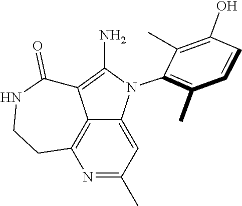

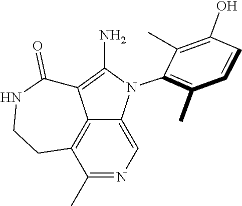

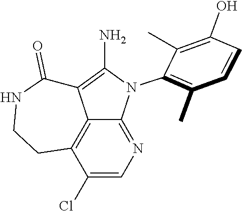

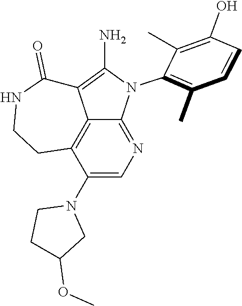

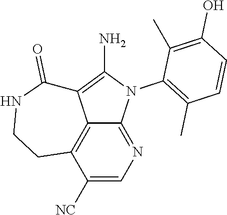 [SMILES: Cc1ccc(O)c(C)c1-n1c(N)c2c3c(c(C#N)cnc31)CCNC2=O]

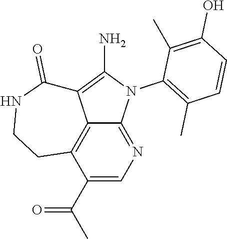 [SMILES: CC(=O)c1cnc2c3c1CCNC(=O)c3c(N)n2-c1c(C)ccc(O)c1C]

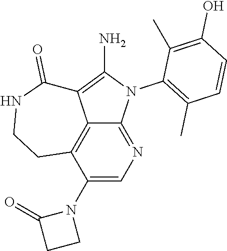 [SMILES: Cc1ccc(O)c(C)c1-n1c(N)c2c3c(c(N4CCC4=O)cnc31)CCNC2=O]

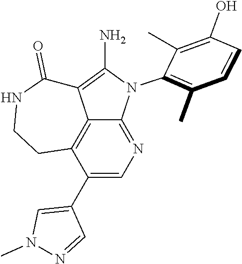

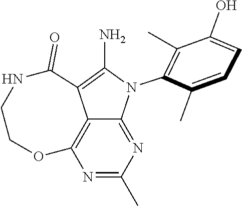

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

In some embodiments, the compounds described herein contain bonds with hindered rotation such that two separate rotamers or atropisomers can be isolated. In some embodiments the atropisomers are

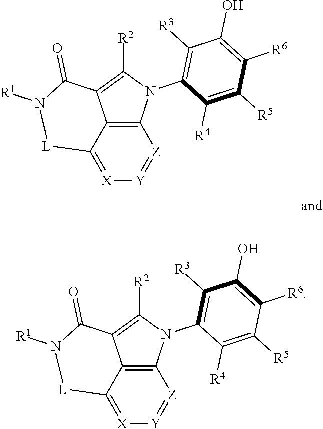

In some embodiments, these atropisomer are separated and are found to have different biological activity which may be advantageous. In some embodiments the atropisomer is

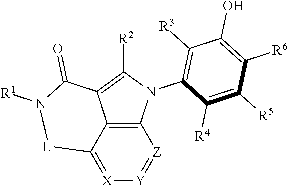

In some embodiments the atropisomer is

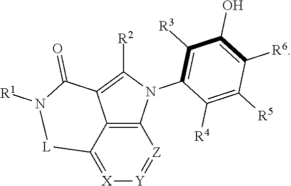

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H (D), ³H, ¹³C, ¹⁴C, ¹⁵N, ¹⁸O, ¹⁷O, ³¹P, ³²p, ³⁵S, ¹F, and ³⁶Cl, respectively. Compounds described herein, and the pharmaceutically acceptable salts thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% of a total number of hydrogen and deuterium. In some embodiments, one or more of the substituents disclosed herein comprise deuterium at a percentage higher than the natural abundance of deuterium. In some embodiments, one or more hydrogens are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein are methods of treating a disease modulated at least in part by PKMYT1 in a subject in need thereof, comprising administering to the subject a therapeutically affective amount of a compound, or a pharmaceutically acceptable salt thereof, disclosed herein.

Disclosed herein is a method of treating cancer in a subject in need thereof, the method comprising administering to the subject a therapeutically affective amount of a compound, or a pharmaceutically acceptable salt thereof, disclosed herein.

In some embodiments, the cancer depends on the activity of PKMYT1.

In some embodiments, the cancer overexpresses CCNE1. In some embodiments cancers which have a high incidence of CCNE1 overexpression include e.g., breast cancer, endometrial cancer, esophageal cancer, lung cancer, ovarian cancer, stomach cancer, and uterine cancer.

In some embodiments, the cancer has an inactivating mutation in the FBXW7 gene. In some embodiments cancers which have a deficiency in FBXW7 include, e.g., breast cancer, colorectal cancer, esophageal cancer, lung cancer, and uterine cancer.

In some embodiments, the cancer is a solid tumor.

In some embodiments, the cancer is breast cancer, colorectal cancer, endometrial cancer, esophageal cancer, glioblastoma, hepatocellular carcinoma, lung cancer, neuroblastoma, ovarian cancer, prostate cancer, stomach cancer, or uterine cancer.

Disclosed herein is a method of modulating PKMYT1 in a subject, the method comprising administering to the subject a compound, or a pharmaceutically acceptable salt thereof, disclosed herein. Disclosed herein is a method of inhibiting PKMYT1 in a subject, the method comprising administering to the subject a compound, or a pharmaceutically acceptable salt thereof, disclosed herein.

Further disclosed herein is a method of selectively inhibiting PKMYT1 in a subject (e.g., selective over WEE1), the method comprising administering to the subject a compound, or a pharmaceutically acceptable salt thereof, disclosed herein.

WEE1 widely exists in human tissues and plays an important role in all phases of the cell cycle. It can be beneficial to use compounds that selectively inhibit PKMYT1 over WEE1 in methods of treatment described herein. In some embodiments, compounds disclosed herein, or a pharmaceutically acceptable salt thereof, do not interact with WEE1. In some embodiments, compounds disclosed herein, or a pharmaceutically acceptable salt thereof, do not inhibit WEE1. In some embodiments, a compound disclosed herein has a WEE1 IC₅₀ value of at least about 100 nM, at least about 500 nM, at least about 1000 nM, or at least about 10,000 nM as determined in a WEE1 ADP-Glo assay. For example, the WEE1 IC₅₀ value can be determined in a WEE1 ADP-Glo assay. In some embodiments, the compound disclosed herein, or a pharmaceutically acceptable salt thereof, has a WEE1 IC₅₀ value of at least about 10,000 nM as determined in a WEE1 ADP-Glo assay. In some embodiments, the compound disclosed herein, or a pharmaceutically acceptable salt thereof, has a WEE1 IC₅₀ value of at least about 5,000 nM as determined in a WEE1 ADP-Glo assay. In some embodiments, the compound disclosed herein, or a pharmaceutically acceptable salt thereof, has a WEE1 IC₅₀ value of at least about 4,000 nM as determined in a WEE1 ADP-Glo assay. In some embodiments, the compound disclosed herein, or a pharmaceutically acceptable salt thereof, has a WEE1 IC₅₀ value of at least about 3,000 nM as determined in a WEE1 ADP-Glo assay. In some embodiments, the compound disclosed herein, or a pharmaceutically acceptable salt thereof, has a WEE1 IC₅₀ value of at least about 2,000 nM as determined in a WEE1 ADP-Glo assay. In some embodiments, the compound disclosed herein, or a pharmaceutically acceptable salt thereof, has a WEE1 IC₅₀ value of at least about 1,000 nM as determined in a WEE1 ADP-Glo assay.

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In some embodiments, doses employed for adult human treatment are typically in the range of 0.01 mg-5000 mg per day. In some embodiments, the daily dosages appropriate for the compound described herein, or a pharmaceutically acceptable salt thereof, are from about 0.01 to about 50 mg/kg per body weight. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

### Combination

Disclosed herein are methods of treating a disease or disorder associated with PKMYT1 using a compound disclosed herein, or a pharmaceutically acceptable salt thereof, in combination with an additional therapeutic agent.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

In some embodiments, the additional therapeutic agent is an anti-cancer agent.

### EXAMPLES

### Example 1: Preparation of 1-amino-2-(3-hydroxy-2,6-dimethylphenyl)-5-methyl-2,6,7,8-tetrahydro-9H-2,3,8-triazabenzo[cd]azulen-9-one

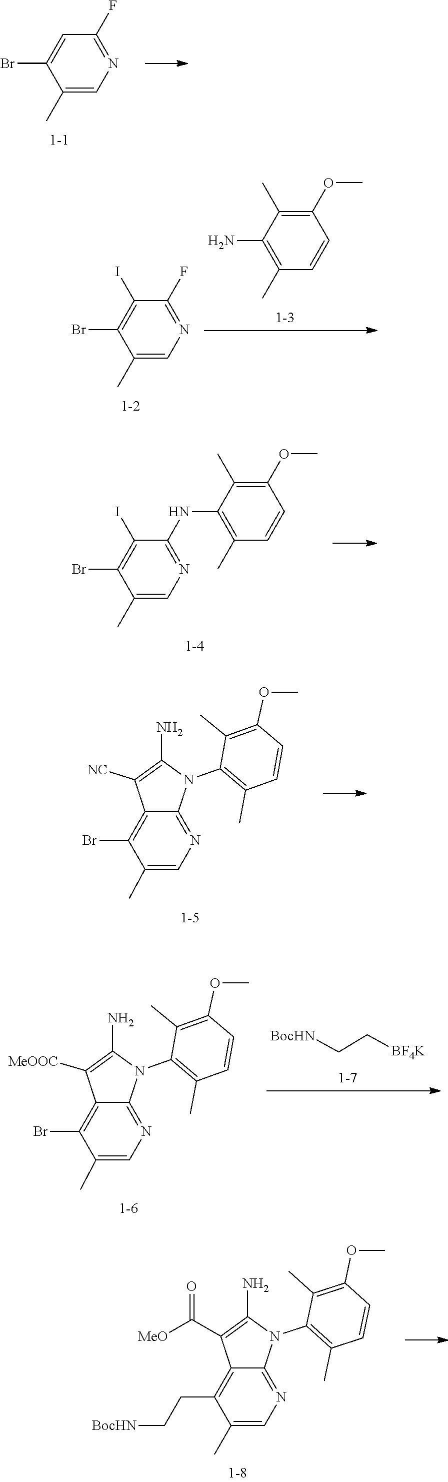

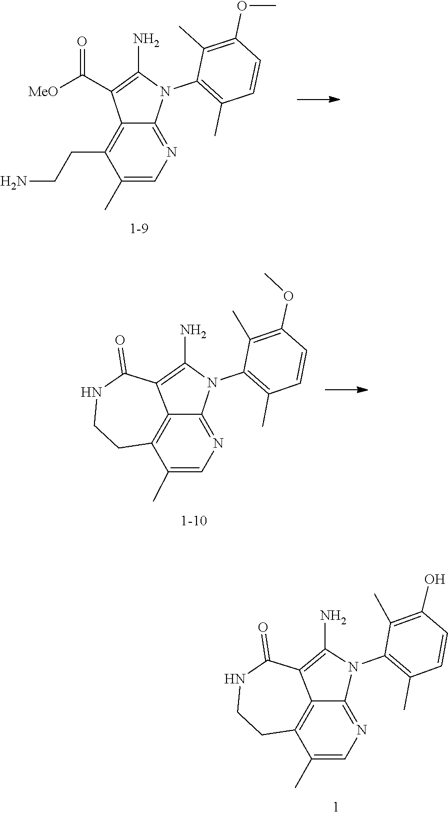 [SMILES: COC(=O)c1c(N)n(-c2c(C)ccc(OC)c2C)c2ncc(C)c(CCN)c12 ; COc1ccc(C)c(-n2c(N)c3c4c(c(C)cnc42)CCNC3=O)c1C ; Cc1ccc(O)c(C)c1-n1c(N)c2c3c(c(C)cnc31)CCNC2=O]

### Step 1: Preparation of Compound 1-2

To a solution of 4-bromo-2-fluoro-5-methylpyridine (3.7 g, 19.47 mmol) in THF (5 mL) was added LDA (11.7 mL, 23.37 mmol) at −78° C. The mixture was stirred at this temperature for 1 h. Then I₂ (5.44 g, 21.419 mmol) was added and stirred at −78° C. for 1 h. The mixture was quenched with NH₄Cl (sat.aq, 30 mL), and extracted with EA (80 mL). The organic layer was washed with saturated Na₂SO₃ (aq, 50 mL), dried over Na₂SO₄, and concentrated to dryness. The residue was purified by silica gel column chromatography to afford 4-bromo-2-fluoro-3-iodo-5-methylpyridine (4.5 g, 73.2%) as a white solid. LCMS: 315.8 [M+H]⁺.

### Step 2: Preparation of Compound 1-4

To a solution of 4-bromo-2-fluoro-3-iodo-5-methylpyridine (5.9 g, 18.68 mmol) and 3-methoxy-2,6-dimethylaniline (2.8 g, 18.68 mmol) in THF (50 mL) was added LiHMDS (1.0 M, 37.4 mL, 37.35 mmol). The mixture was stirred at 25° C. for 2 hours. The reaction mixture was quenched by NH₄Cl (100 mL), and extracted with EA (100 mL×2). The organic layer were combined and dried over Na₂SO₄. After filtration, the filtrate was concentrated and purified by silica gel column chromatography to afford 4-bromo-3-iodo-2-[(3-methoxy-2,6-dimethylphenyl)amino]-5-methylpyridine (7.0 g, 83.8%) as a yellow solid. LCMS: 448.9 [M+H]⁺.

### Step 3: Preparation of Compound 1-5

To a solution of propanedinitrile (1.1 g, 16.24 mmol) in DME (10 mL) was added NaH (0.65 g, 16.24 mmol) at 0° C. The mixture was stirred at 25° C. for 0.5 h. Then XantPhos Pd G3 (0.70 g, 0.81 mmol) and 5-chloro-4-[(3-methoxy-2,6-dimethylphenyl)amino]-2-methylpyrimidine (6.6 g, 14.76 mmol) was added. The mixture was stirred at 100° C. for 5 hours. The mixture was filtered. The filtrate was concentrated and purified by silica gel column chromatography to afford 2-amino-4-bromo-1-(3-methoxy-2,6-dimethylphenyl)-5-methylpyrrolo[2,3-b]pyridine-3-carbonitrile (5.10 g, 89.7%) as a yellow solid. LCMS: 387.0 [M+H]⁺.

### Step 4: Preparation of Compound 1-6

To a solution of 2-amino-4-bromo-1-(3-methoxy-2,6-dimethylphenyl)-5-methylpyrrolo[2,3-b]pyridine-3-carbonitrile (3.0 g, 7.79 mmol) in Methanol (30 mL) was added H₂SO₄ (10 mL). The mixture was heated to 100° C. for 16 hours. The mixture was poured into ice-water, which was adjusted to pH˜8 with NaOH (4M in water). The mixture was extracted with dichloromethane (50 mL). The organic layer was dried over Na₂SO₄, filtered, concentrated, and purified by silica gel column chromatography to afford methyl 2-amino-4-bromo-1-(3-methoxy-2,6-dimethylphenyl)-5-methylpyrrolo[2,3-b]pyridine-3-carboxylate (0.30 g, 9.2%) as a yellow solid. LCMS: 419.1 [M+H]⁺.

### Step 5: Preparation of Compound 1-8

To a solution of methyl 2-amino-4-bromo-1-(3-methoxy-2,6-dimethylphenyl)-5-methyl-1H-pyrrolo[2,3-b]pyridine-3-carboxylate (0.30 g, 0.72 mmol) and potassium (2-((tert-butoxycarbonyl)amino)ethyl)trifluoroborate (0.539 g, 2.15 mmol) in dioxane (5 mL) and water (0.5 mL) was added Pd(dtbpf)Cl₂ (0.047 g, 0.07 mmol) and K₃PO₄ (0.456 g, 2.15 mmol). The mixture was stirred at 130° C. for 1 h (microwave). The reaction mixture was cooled to room temperature, diluted with EA (30 mL), and washed with brine (30 mL). The organic layer was dried over Na₂SO₄, filtered, and concentrated to dryness. The residue was purified by Pre-TLC to afford methyl 2-amino-4-(2-((tert-butoxycarbonyl)amino)ethyl)-1-(3-methoxy-2,6-dimethylphenyl)-5-methyl-1H-pyrrolo[2,3-b]pyridine-3-carboxylate (0.060 g, 17.4%) as a yellow solid. LCMS: 483.3 [M+H]⁺.

### Step 6: Preparation of Compound 1-9

To a solution of 2-methylpropan-2-yl ({2-[2-amino-3-(methoxycarbonyl)-1-(3-methoxy-2,6-dimethylphenyl)-5-methylpyrrolo[2,3-b]pyridin-4-yl]ethyl}amino)methanoate (0.060 g, 0.12 mmol) in dichloromethane (2 mL) was added TFA (0.141 g, 1.2 mmol). The mixture was stirred at 15° C. for 1 h. The reaction mixture was diluted with dichloromethane (10 mL), and washed with NaHCO₃ (aq., 10 mL). The organic layer was dried over Na₂SO₄, filtered, and concentrated to dryness. The residue was purified by Pre-TLC to afford methyl 2-amino-4-(2-aminoethyl)-1-(3-methoxy-2,6-dimethylphenyl)-5-methylpyrrolo[2,3-b]pyridine-3-carboxylate (0.033 g, 69.4%) as a yellow solid. LCMS: 383.2 [M+H]⁺.

### Step 7: Preparation of Compound 1-10

To a solution of methyl 2-amino-4-(2-aminoethyl)-1-(3-methoxy-2,6-dimethylphenyl)-5-methylpyrrolo[2,3-b]pyridine-3-carboxylate (0.015 g, 0.04 mmol) in dioxane (1 mL) was added trimethylaluminum (0.39 mL, 0.78 mmol) (2M in toluene). The mixture was stirred at 100° C. for 18 h. The reaction mixture was quenched by methanol (1 mL), and filtered. The filtrate was concentrated to dryness and purified by Pre-TLC to afford 3-amino-2-(3-methoxy-2,6-dimethylphenyl)-10-methyl-2,6,12-triazatricyclo[7.3.1.0^{4,13}]trideca-1(13),3,9(10),11-tetraen-5-one as a yellow solid. LCMS: 351.2 [M+H]⁺.

### Step 8: Preparation of Example 1

To a solution of 3-amino-2-(3-methoxy-2,6-dimethylphenyl)-10-methyl-2,6,12-triazatricyclo[7.3.1.0^{4,13}]trideca-1(13),3,9(10),11-tetraen-5-one (0.004 g, 0.011 mmol) in dichloromethane (1 mL) was added BBr₃ (14 mg, 0.057 mmol) at 0° C. The mixture was stirred for 0.5 h. The reaction was quenched with methanol (1 mL), and concentrated to dryness. The residue was purified by Pre-HPLC to afford 1-amino-2-(3-hydroxy-2,6-dimethylphenyl)-5-methyl-2,6,7,8-tetrahydro-9H-2,3,8-triazabenzo[cd]azulen-9-one as a yellow solid. LCMS: 337.1 [M+H]⁺. ¹HNMR: (400 MHz, CD₃OD) δ 7.57 (s, 1H), 7.10 (d, J=8.4 Hz, 1H), 6.91 (d, J=8.4 Hz, 1H), 3.59 (d, J=5.1 Hz, 2H), 3.12 (s, 2H), 2.28 (s, 3H), 1.87 (s, 3H), 1.82 (s, 3H).

### Example 2: Preparation of 1-amino-2-(3-hydroxy-2,6-dimethylphenyl)-5-methyl-2,6,7,8-tetrahydro-9H-2,4,8-triazabenzo[cd]azulen-9-one

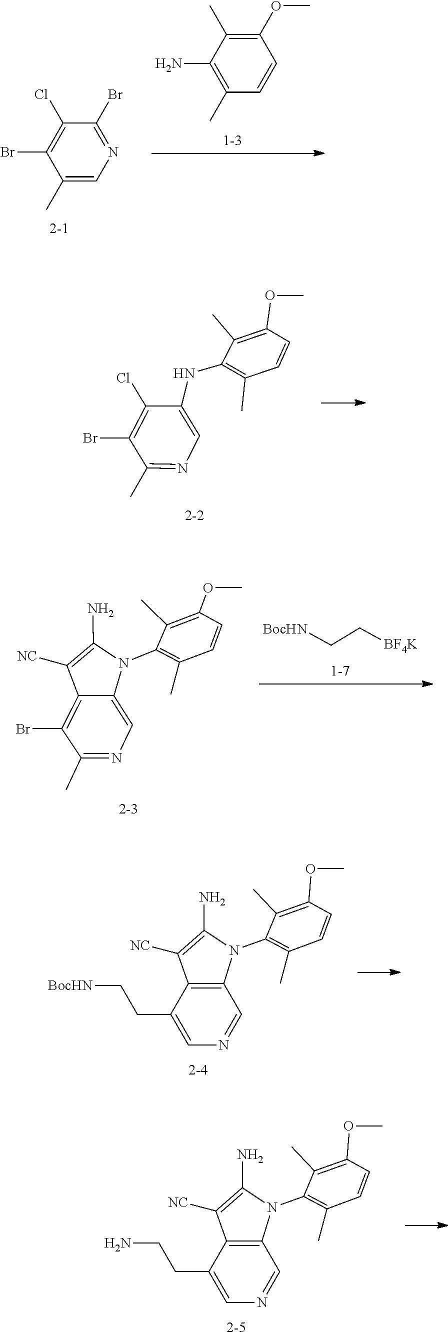

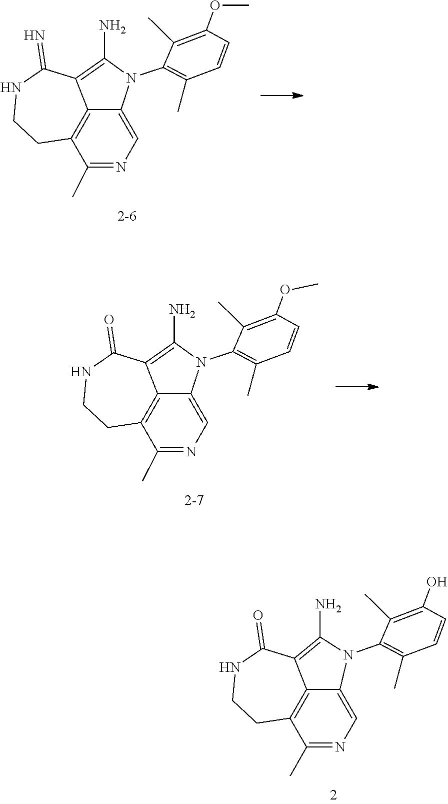 [SMILES: COc1ccc(C)c(-n2c(N)c3c4c(c(C)ncc42)CCNC3=N)c1C ; COc1ccc(C)c(-n2c(N)c3c4c(c(C)ncc42)CCNC3=O)c1C ; Cc1ccc(O)c(C)c1-n1c(N)c2c3c(c(C)ncc31)CCNC2=O]

### Step 1: Preparation of Compound 2-2

A mixture of compound 2-1 (3.7 g, 12.966 mmol), compound 1-3 (2.0 g, 12.966 mmol), Pd₂(dba)₃ (1.2 g, 1.297 mmol), Xantphos (1.5 g, 2.593 mmol) and Cs₂CO₃ (12.7 g, 38.898 mmol) in dioxane (2 mL) was heated to 110° C. for 16 h under N₂. The mixture was concentrated, and the residue was purified by column chromatography on silica gel to afford a crude product. The crude product was purified by column chromatography to afford compound 2-2 (2.7 g, 58.6%) as a white solid. LCMS: 357.0 [M+H]⁺.

### Step 2: Preparation of Compound 2-3

To a solution of propanedinitrile (0.89 g, 13.496 mmol) in NMP (30 mL) was added NaH (0.36 g, 14.846 mmol) in portions at 0° C. under N₂. The mixture was stirred at rt for 1 h. Then compound 2-2 (2.4 g, 6.748 mmol) was added. The mixture was heated to 130° C. for 1 h in a microwave under N₂. The mixture was quenched by H₂O (50 mL) and extracted with EA (50 mL×3). Combined organic layers were washed with brine, dried over Na₂SO₄, filtered and concentrated. The residue was purified by column chromatography on silica gel to afford compound 2-3 (2.1 g, 80.8%) as a yellow solid. LCMS: 385.1 [M+H]⁺.

### Step 3: Preparation of Compound 2-4

A mixture of compound 2-3 (1.5 g, 3.893 mmol), compound 1-7 (2.1 g, 1.787 mmol), Cs₂CO₃ (3.8 g, 11.680 mmol) and Ruphos Pd G₄ (0.33 g, 0.389 mmol) in dioxane-water (5:1) (40 mL) was heated to 105° C. for 16 h under N₂. The mixture was diluted with H₂O (50 mL), extracted with EA (50 mL×3). Combined organic layers were washed with brine, dried over Na₂SO₄, filtered and concentrated. The residue was purified by column chromatography on silica gel to afford compound 2-4 (600 mg, 34.3%) as a yellow solid. LCMS: 450.4 [M+H]⁺.

### Step 4: Preparation of Compound 2-5

A mixture of 2 compound 2-4 (600 mg, 1.335 mmol) in DCM (9 mL) and TFA (3 mL) was stirred at rt for 2 h. The mixture was concentrated, and the residue was diluted with NaHCO₃ (sat.aq, 10 mL), extracted with EA (10 mL×4). The combined organic layers were washed with brine, dried over Na₂SO₄, filtered and concentrated. The residue was purified by column chromatography on silica gel to afford compound 2-5 (420 mg, 90.1%) as a yellow solid. LCMS: 350.2 [M+H]⁺.

### Step 5: Preparation of Compound 2-6

A mixture of compound 2-5 (100 mg, 0.286 mmol) in HOAc (1 mL) and EtOH (1 mL) was heated to 110° C. for 16 h. The mixture was concentrated, and the residue was diluted with NaHCO₃ (sat. aq, 10 mL), extracted with EA (10 mL×3). Combined organic layers were washed with brine, dried over Na₂SO₄, filtered and concentrated. The residue was purified by prep-TLC to afford compound 2-6. LCMS: 350.2 [M+H]⁺.

### Step 6: Preparation of Compound 2-7

A mixture of compound 2-6 (100 mg, 0.286 mmol) and NaOH (2.9 mL, 2.862 mmol) in EtOH (0.6 mL) was heated to 100° C. for 16 h. The mixture was concentrated, and the residue was diluted with H₂O (10 mL), extracted with EA (10 mL×3). Combined organic layers were washed with brine, dried over Na₂SO₄, filtered and concentrated. The residue was purified by prep-TLC to afford compound 2-7. LCMS: 351.3 [M+H]⁺.

### Step 7: Preparation of Example 2

To a solution of compound 2-7 (20 mg, 0.057 mmol) in DCM (1 mL) was added BBr₃ (0.6 mL, 0.571 mmol) in drops. The mixture was stirred at rt for 1 h. The mixture was concentrated, and the residue was purified by prep-HPLC to afford Example 2. LCMS: 337.1 [M+H]⁺; ¹H NMR: (400 MHz, CD₃OD) δ 8.43 (s, 1H), 7.47 (s, 1H), 7.07 (d, J=8.3 Hz, 1H), 6.89 (d, J=8.3 Hz, 1H), 3.52 (d, J=4.2 Hz, 2H), 3.09 (s, 2H), 2.48 (s, 3H), 1.80 (s, 3H), 1.74 (s, 3H).

### Example 3: Preparation of 1-amino-2-(3-hydroxy-2,6-dimethylphenyl)-4-methyl-2,6,7,8-tetrahydro-9H-2,3,5,8-tetraazabenzo[cd]azulen-9-one

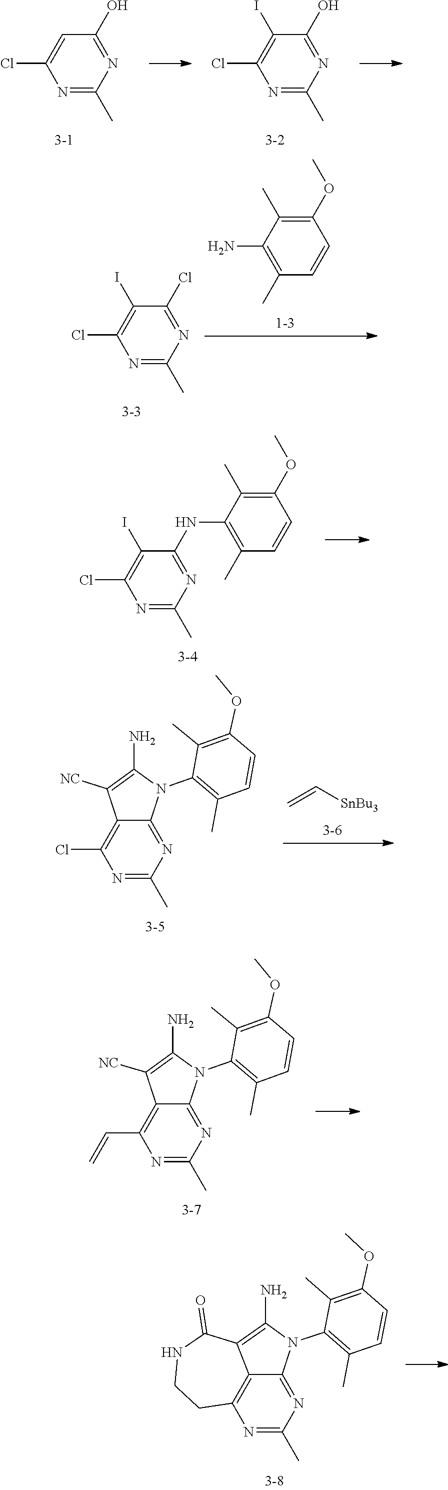

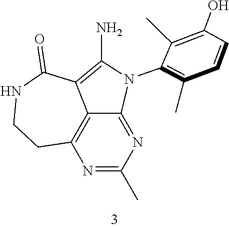

### Step 1: Preparation of Compound 3-2

To a solution of compound 3-1 (5 g, 34.587 mmol) in DMF (50 mL) was added NIS (15.6 g, 69.333 mmol). The mixture was stirred at 70° C. for 2 h. The mixture was diluted with water (250 mL) and sat.aq Na₂S₂O₃ (250 mL), extracted with EA (200 mL×3). The combined organic layers were washed with water (500 mL) and brine (500 mL), dried over Na₂SO₄, filtered, and concentrated to dryness. The residue was purified by column chromatography on silica gel to afford compound 3-2 (8.7 g, 93.0%) as a yellow solid. LCMS: 270.8 [M+H]⁺.

### Step 2: Preparation of Compound 3-3

To a solution of compound 3-2 (6.7 g, 24.769 mmol) in POCl₃ (70 mL) was stirred at 105° C. for 1 h. The reaction mixture was concentrated and diluted with EA (200 mL), which was washed with water (200 mL×3), dried over Na₂SO₄, filtered, and concentrated to dryness. The residue was purified by silica gel column chromatography to afford compound 3-3 (6.2 g, 86.6%) as a white solid. LCMS: 288.7 [M+H]⁺.

### Step 3: Preparation of Compound 3-4

To a solution of compound 3-3 (6.2 g, 21.461 mmol) and compound 1-3 (3.9 g, 25.794 mmol) in THF (100 mL) was added LiHMDS (42.9 mL, 42.922 mmol) at 0° C. The mixture was stirred at 0° C. for 1 h. The reaction mixture was carefully added into NH₄Cl (aq, 100 mL) under ice-bath, which was extracted with EA (100 mL×3). The combined organic layers were washed with brine (50 mL×3), dried over Na₂SO₄, filtered, and concentrated to dryness. The residue was purified by silica gel column chromatography to afford compound 3-4 (2.15 g, 24.8%) as a yellow solid. LCMS: 404.0 [M+H]⁺.

### Step 4: Preparation of Compound 3-5

To a solution of propanedinitrile (818.8 mg, 12.387 mmol) in DME (40 mL) was added NaH (495.5 mg, 12.388 mmol) at 0° C., the mixture was stirred at 0° C. for 30 min. Then to the mixture was added compound 3-4 (2.0 g, 4.955 mmol) and XantPhos Pd G₃ (470.4 mg, 0.495 mmol), the mixture was stirred at 130° C. for 4 h. The reaction mixture was diluted with water (300 mL), which was extracted with EA (300 mL×3). The combined organic layers were washed with brine (500 mL×2), dried over Na₂SO₄, filtered, and concentrated to dryness. The residue was purified by silica gel column chromatography to afford compound 3-5 (700 mg, 41.3%) as a white solid. LCMS: 342.2 [M+H]⁺.

### Step 5: Preparation of Compound 3-7

To a solution of compound 3-5 (200 mg, 0.585 mmol) in THF (8 mL) were added compound 3-6 (371.0 mg, 1.170 mmol) and Pd(PPh₃)₂Cl₂ (41.1 mg, 0.0585 mmol). The mixture was stirred at 100° C. for 16 h. The reaction mixture was diluted with water (40 mL), which was extracted with EA (40 mL×3). The combined organic layer was washed with brine (40 mL), dried over Na₂SO₄, filtered, and concentrated to dryness. The residue was purified by silica gel column chromatography and prep-HPLC to afford compound 3-7. LCMS: 334.3 [M+H]⁺.

### Step 6: Preparation of Compound 3-8

To a solution of compound 3-7 (110 mg, 0.330 mmol) in chlorobenzene (4 mL) was added pTSA (0.07 mL, 0.988 mmol). The mixture was stirred at 100° C. for 4 h. The mixture was concentrated to dryness. The residue was purified by silica gel column chromatography to afford compound 3-8. LCMS: 352.1 [M+H]⁺.

### Step 7: Preparation of Example 3

To a solution of compound 3-8 (57 mg, 0.162 mmol) in DCM (5 mL) was added BBr₃ (203 mg, 0.810 mmol) at 0° C. The mixture was stirred at rt for 1 h. The mixture was quenched by MeOH (3 mL) while stirring and was diluted with water (10 mL), which was adjusted to pH˜8 with NaOH (4 M). Then the mixture was extracted with DCM (10 mL×3). The combined organic layer was dried over Na₂SO₄, filtered, and concentrated to dryness. The residue was separated by prep-SFC and chiral HPLC to afford Example 3. LCMS: 338.2 [M+H]⁺; ¹H NMR: (400 MHz, DMSO-d₆) δ 9.61 (s, 1H), 7.22 (t, J=4.5 Hz, 1H), 7.08 (d, J=8.4 Hz, 1H), 6.94 (d, J=8.3 Hz, 1H), 6.89 (s, 2H), 3.45 (dd, J=9.8, 4.6 Hz, 2H), 3.06-3.02 (m, 2H), 2.41 (s, 3H), 1.78 (s, 3H), 1.69 (s, 3H).

### Example A: PKMYT1 HTRF Assay

Compound serial dilution is performed by Echo, and the final concentrations vary from 10 μM to 0.5 nM. This was filled by the addition of 5 μL/well of Enzyme solution to the assay plate containing the compound. The plate was centrifuged at 1000 rpm for 1 minute, and incubate 15 minutes at 25° C. Then 5 μL/well of tracer solution (Tracer 178) was added to initiate the reaction, and incubate for 60 minutes at 25° C. Next 5 μL GST-Tb was added into the assay plate, the plate was centrifuged at 1000 rpm for 1 minute, and incubate for 15 minutes at 25° C. The assay plate was read on Envision.

### Example B: WEE1 ADP-Glo Assay

Compound serial dilution is performed by Echo, and the final concentrations vary from 10 μM to 0.5 nM. This was filled by the addition of 5 μL/well of Enzyme solution to the assay plate containing the compound. The plate was centrifuged at 1000 rpm for 1 minute, and incubate 15 minutes at 25° C. Then 5 μL/well of substrate solution was added to initiate the reaction, and incubate for 60 minutes at 25° C. Next 10 μL kinase detection reagent was added into the assay plate, the plate was centrifuged at 1000 rpm for 1 minute, and incubate for 60 minutes at 25° C. The assay plate was read on Envision for US LUM as RLU.

The data for Example A and Example B is shown in Table 3.

TABLE 3

| Ex. | MYT1 HTRF IC₅₀ (nM) | WEE1 ADP-Glo IC₅₀ (nM) |
|---|---|---|
| 1 | A | 1206 |
| 2 | B | >10000 |
| 3 | A | Not tested |

MYT1 HTRF IC₅₀ (nM): 0 < A ≤ 10; 10 < B ≤ 50; 50 < C ≤ 500; 500 < D ≤ 5000; 5000 < E

What is claimed is:

1. A compound of Formula (I), or a pharmaceutically acceptable salt thereof: 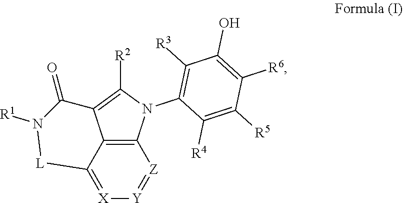
wherein
R¹ is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
L is C₁-C₄alkylene or C₁-C₄heteroalkylene comprising 1 or 2 heteroatoms selected from the group consisting of 0, N, and S; each optionally substituted with one or more R⁷;
each R⁷ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁷ on the same atom are taken together to form an oxo;
or two R⁷ on adjacent carbons are taken together to form an alkenylene;
or two R⁷ on the same atom or different atoms are taken together to form a cycloalkyl, heterocycloalkyl, aryl, or heteroaryl, each optionally substituted with one or more R;
R² is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Xa};
each R^{Xa} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Xa} on the same atom are taken together to form an oxo;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Ya};
each R^{Ya} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Ya} on the same atom are taken together to form an oxo;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
R³ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁴ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, or C₁-C₆heteroalkyl, or C₃-C₆cycloalkyl;
or two R on the same atom form an oxo.

2. The compound of claim 1, or a pharmaceutically acceptable salt thereof, wherein X is N.

3. The compound of claim 1, or a pharmaceutically acceptable salt thereof, wherein X is CR^{X}.

4. The compound of any one of claims 1-3, or a pharmaceutically acceptable salt thereof, wherein Y is N.

5. The compound of any one of claims 1-3, or a pharmaceutically acceptable salt thereof, wherein Y is CR^{Y}.

6. The compound of any one of claims 1-5, or a pharmaceutically acceptable salt thereof, wherein Z is N.

7. The compound of any one of claims 1-5, or a pharmaceutically acceptable salt thereof, wherein Z is CR^{Z}.

8. The compound of claim 1, or a pharmaceutically acceptable salt thereof, wherein the compound is of Formula (Ia):

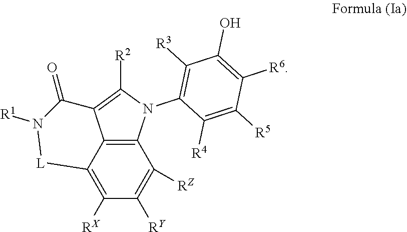

9. The compound of claim 1, or a pharmaceutically acceptable salt thereof, wherein the compound is of Formula (Ib):

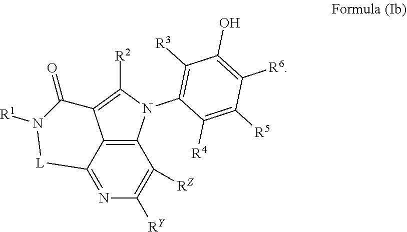

10. The compound of claim 1, or a pharmaceutically acceptable salt thereof, wherein the compound is of Formula (Ic):

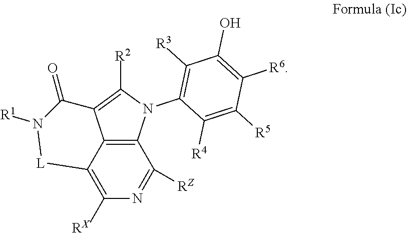

11. The compound of claim 1, or a pharmaceutically acceptable salt thereof, wherein the compound is of Formula (Id):

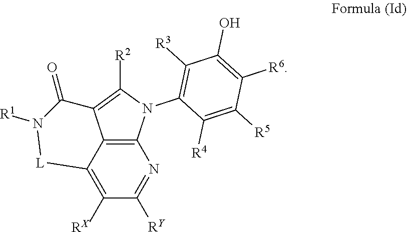

12. The compound of claim 1, or a pharmaceutically acceptable salt thereof, wherein the compound is of Formula (Ie):

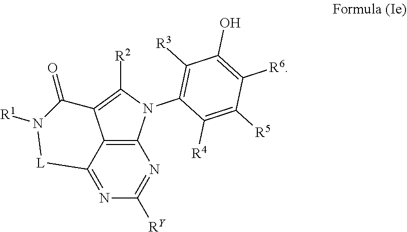

13. The compound of any one of claims 1-12, or a pharmaceutically acceptable salt thereof, wherein R³ is halogen, C₁-C₆alkyl, or C₁-C₆haloalkyl.

14. The compound of any one of claims 1-13, or a pharmaceutically acceptable salt thereof, wherein R³ is C₁-C₆alkyl.

15. The compound of any one of claims 1-14, or a pharmaceutically acceptable salt thereof, wherein R⁴ is halogen, C₁-C₆alkyl, or C₁-C₆haloalkyl.

16. The compound of any one of claims 1-15, or a pharmaceutically acceptable salt thereof, wherein R⁴ is C₁-C₆alkyl.

17. The compound of any one of claims 1-16, or a pharmaceutically acceptable salt thereof, wherein R⁵ is hydrogen.

18. The compound of any one of claims 1-17, or a pharmaceutically acceptable salt thereof, wherein R⁶ is hydrogen.

19. The compound of any one of claims 1-18, or a pharmaceutically acceptable salt thereof, wherein R² is —NR^{c}R^{d}.

20. The compound of any one of claims 1-19, or a pharmaceutically acceptable salt thereof, wherein R^{X} is hydrogen, halogen, —CN, —OH, —OR^{a}, —NR^{c}R^{d}, —C(═O)R^{a}, C₁-C₆alkyl, C₁-C₆haloalkyl, cycloalkyl, heterocycloalkyl, aryl, or heteroaryl; wherein the alkyl, cycloalkyl, heterocycloalkyl, aryl, and heteroaryl is optionally substituted with one or more R^{Xa}.

21. The compound of any one of claims 1-20, or a pharmaceutically acceptable salt thereof, wherein R^{X} is hydrogen, halogen, —CN, —C(═O)R^{a}, C₁-C₆alkyl, heterocycloalkyl, or heteroaryl; wherein the alkyl, heterocycloalkyl, and heteroaryl is optionally substituted with one or more R^{Xa}.

22. The compound of any one of claims 1-21, or a pharmaceutically acceptable salt thereof, wherein R^{X} is hydrogen, halogen, or C₁-C₆alkyl.

23. The compound of any one of claims 1-22, or a pharmaceutically acceptable salt thereof, wherein R^{X} is C₁-C₆alkyl.

24. The compound of any one of claims 1-23, or a pharmaceutically acceptable salt thereof, wherein R^{Y} is hydrogen, halogen, —CN, —OH, C₁-C₆alkyl, C₁-C₆haloalkyl, cycloalkyl, or heterocycloalkyl;
wherein the alkyl, cycloalkyl, and heterocycloalkyl is optionally substituted with one or more R^{Ya}.

25. The compound of any one of claims 1-24, or a pharmaceutically acceptable salt thereof, wherein R^{Y} is hydrogen, halogen, C₁-C₆alkyl, or C₁-C₆haloalkyl.

26. The compound of any one of claims 1-25, or a pharmaceutically acceptable salt thereof, wherein R^{Y} is hydrogen.

27. The compound of any one of claims 1-26, or a pharmaceutically acceptable salt thereof, wherein R^{Z} is hydrogen, halogen, —CN, —OH, C₁-C₆alkyl, C₁-C₆haloalkyl, cycloalkyl, or heterocycloalkyl.

28. The compound of any one of claims 1-27, or a pharmaceutically acceptable salt thereof, wherein R^{Z} is hydrogen, halogen, C₁-C₆alkyl, or C₁-C₆haloalkyl.

29. The compound of any one of claims 1-28, or a pharmaceutically acceptable salt thereof, wherein R^{Z} is hydrogen.

30. The compound of any one of claims 1-29, or a pharmaceutically acceptable salt thereof, wherein R¹ is hydrogen or C₁-C₆alkyl.

31. The compound of any one of claims 1-30, or a pharmaceutically acceptable salt thereof, wherein R^{Y} is hydrogen.

32. The compound of any one of claims 1-31, or a pharmaceutically acceptable salt thereof, wherein L is C₁-C₄alkylene optionally substituted with one or more R⁷.

33. The compound of any one of claims 1-31, or a pharmaceutically acceptable salt thereof, wherein L is C₁-C₃alkylene optionally substituted with one or more R⁷.

34. The compound of any one of claims 1-31, or a pharmaceutically acceptable salt thereof, wherein L is C₂alkylene optionally substituted with one or more R⁷.

35. The compound of any one of claims 1-31, or a pharmaceutically acceptable salt thereof, wherein L is C₁-C₄heteroalkylene comprising 1 or 2 heteroatoms selected from the group consisting of O, N, and S; optionally substituted with one or more R⁷.

36. The compound of any one of claims 1-31, or a pharmaceutically acceptable salt thereof, wherein L is C₁-C₂heteroalkylene comprising 1 heteroatom selected from the group consisting of O, N, and S; optionally substituted with one or more R⁷.

37. The compound of any one of claims 1-31, or a pharmaceutically acceptable salt thereof, wherein L is C₁-C₂heteroalkylene comprising 1 heteroatom selected from the group consisting of O and N; optionally substituted with one or more R⁷.

38. The compound of any one of claims 1-31, or a pharmaceutically acceptable salt thereof, wherein L is C₁-C₂heteroalkylene comprising 1 heteroatom that is O; optionally substituted with one or more R⁷.

39. The compound of any one of claims 1-38, or a pharmaceutically acceptable salt thereof, wherein each R⁷ is independently halogen, —CN, —OH, —OR^{a}, or —NR^{c}R^{d}.

40. The compound of any one of claims 1-38, or a pharmaceutically acceptable salt thereof, wherein each R⁷ is independently halogen.

41. The compound of claim 1, or a pharmaceutically acceptable salt thereof, wherein the compound is selected from a compound found in table 1 or table 2.

42. A pharmaceutical composition comprising the compound of any one of claims 1-41, or a pharmaceutically acceptable salt thereof, and a pharmaceutically acceptable excipient.

43. A method of treating cancer in a subject in need thereof, the method comprising administering to the subject the compound of any one of claims 1-41, or a pharmaceutically acceptable salt thereof.

44. A method of modulating PKMYT1 in a subject, the method comprising administering to the subject the compound of any one of claims 1-41, or a pharmaceutically acceptable salt thereof.

45. A method of inhibiting PKMYT1 in a subject, the method comprising administering to the subject the compound of any one of claims 1-41, or a pharmaceutically acceptable salt thereof.

46. The method of claim 44 or 45, wherein the compound or a pharmaceutically acceptable salt thereof does not inhibit WEE1.

47. The method of any one of claims 44-46, wherein the subject has cancer.

48. The method of claim 43 or 47, wherein the cancer depends on the activity of PKMYT1.

49. The method of any one of claim 43 or 47 or 48, wherein the cancer overexpresses CCNE1.

50. The method of any one of claim 43 or 47-49, wherein the cancer has an inactivating mutation in the FBXW7 gene.

51. The method of any one of claim 43 or 47-50, wherein the cancer is a solid tumor.

52. The method of any one of claim 43 or 47-51, wherein the cancer is breast cancer, colorectal cancer, endometrial cancer, esophageal cancer, glioblastoma, hepatocellular carcinoma, lung cancer, neuroblastoma, ovarian cancer, prostate cancer, stomach cancer, or uterine cancer.
